(19) United States
(12) Patent Application Publication
(10) Pub. No.: US 2013/0272974 A1
(43) Pub. Date: Oct. 17, 2013

(54) Hydroxytyrosol Product, Method of Making, and Uses Thereof

(71) Applicant: Ahmad Alkayali, Murrieta, CA (US)

(72) Inventor: Ahmad Alkayali, Murrieta, CA (US)

(21) Appl. No.: 13/445,092

(22) Filed: Apr. 12, 2012

(51) Int. Cl. A61K 8/34; A61Q 1/06; A61K 31/05; A61Q 17/04; C12S 3/00

(52) U.S. Cl. 424/59; 424/64; 435/267; 514/734

(57) ABSTRACT

The present invention provides a granule powder product containing a minimum of 25% Hydroxytyrosol, and a method of making such a product. The product is made from cultivated olive leaves. Because of unique production and purification technology provided by the present invention, Oleuropein present in the olive leaf extract is essentially completely transferred into Hydroxytyrosol by a biotransformation process. As a result, essentially no Oleuropein residue is present in the final product.

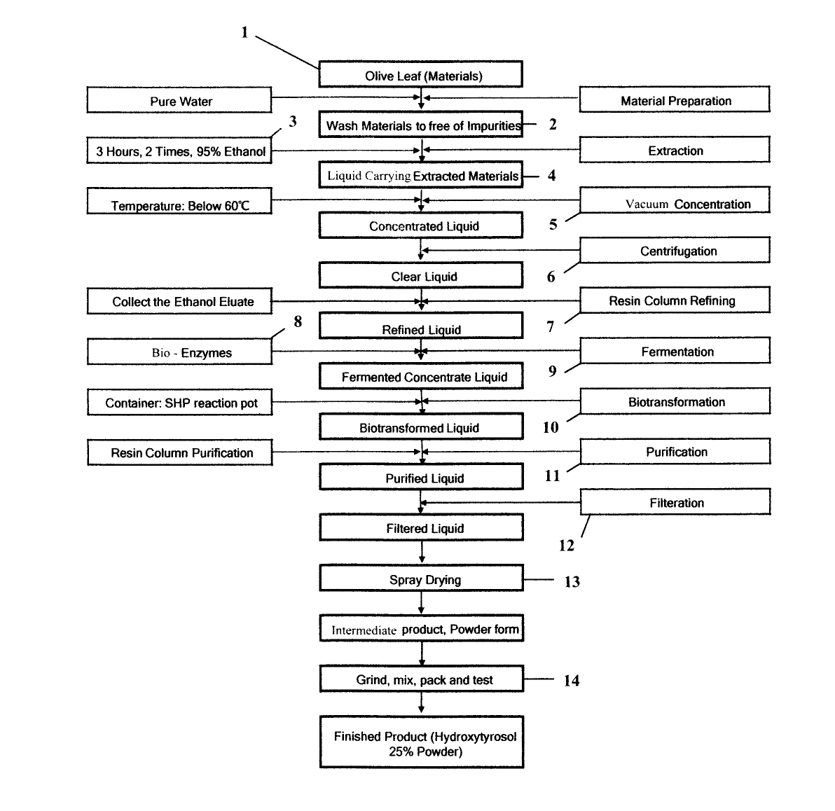

### FIELD OF THE INVENTION

The present invention relates to a method of preparing Hydroxytyrosol in a dry powder product containing as much as 25% Hydroxytyrosol, to the product so produced as presented for human oral consumption as a therapeutic, dietary or nutritional food supplement, and to uses of this product as a therapeutic agent, dietary, and nutritional supplement.

### BACKGROUND OF THE INVENTION

Hydroxytyrosol (3,4-dihydroxyphenylethanol; DOPET) is a phytochemical with antioxidant properties and is the most powerful antioxidants discovered to date. Its Oxygen Radical Absorbance Capacity (ORAC) is 40,000 umolTE/g, which is considered to be ten times higher than Green Tea, and two times higher than CoQ10.

Hydroxytyrosol is naturally found in Olive pulps, leaves, and Olive Oil, especially Extra Virgin Olive Oil. Olive oil is also a source of at least 30 phenolic compounds. The major compounds are Oleuropein, Hydroxytyrosol, and Tyrosol. Recently, a surge of scientific research and published reports reveals the biological properties of Hydroxytyrosol, a strong antioxidant and free-radicals scavenger. And, the medicinal benefits. Olive “waste water” is recognized to contain also compounds with strong antioxidant properties; typically, Hydroxytyrosol which is superior to oleuropein, and tyrosol.

Published research and studies confirms that Hydroxytyrosol may help reduce the risk of heart disease. Hydroxytyrosol showed strong inhibition of lipid peroxidation, which was 6 times stronger than oleuropein. Oxidative damage of low-density lipoproteins increases the risk for atherosclerosis and heart disease. Isoprostanes are markers of lipid peroxidation and are produced from arachidonic acid after free radical injury and have strong biological activities. Hydroxytyrosol from olive leaf extract experimentally and shown anti-HIV activity by blocking the HIV virus from entry into host cells. Hydroxytyrosol also appears to reduce inflammatory indicators, and may represent a natural agent for the control of inflammatory genes.

The recognized advantages of Hydroxytyrosol consumption have resulted in “home remedy” methods for obtaining this material from olive leaves. According to these home remedy methods, a significant advantage can be obtained by simply steeping olive leaves in water at a temperature between 175° and 185° F., and then drinking the resulting “olive leaf tea.” However, such methods are believed to result in only a weak solution of Hydroxytyrosol and may include other constituents of the grape leaf or even surface contaminants present on the leaves, which a person would not want to consume. Further, some people find the olive leaf tea to be unpalatable.

A better way of obtaining high-potency Hydroxytyrosol from olive leaves in an easily consumed palatable form is clearly needed.

### SUMMARY OF THE INVENTION

One embodiment of the present invention is a granule powder product containing about 25% Hydroxytyrosol. It is made from cultivated olive leaves. Due to the unique production and purification technology provided by the present invention, Oleuropein present in the olive leaf extract has been completely transferred into Hydroxytyrosol by a biotransformation process, and essentially no Oleuropein residue is present in final product.

The present invention also provides a method of extracting Hydroxytyrosol from olive leaves in order to provide a dry powder product containing about 25% Hydroxytyrosol, said method comprising in order the steps of: soaking olive leaves in a solution of ethanol to obtain a liquid carrying extracted materials including Hydroxytyrosol and Oleuropein; concentrating the liquid carrying extracted materials to reduce its volume; adding bio-enzymes to the concentrated liquid carrying extracted materials, and utilizing the bio-enzymes to effect fermentation of the concentrated liquid carrying extracted materials; adding biological invertase, and utilizing the biological invertase in a bio-reactor to effect transformation of the Oleuropein in the liquid substantially entirely into Hydroxytyrosol; spray drying the liquid resulting from the prior step to produce a dry powder product substantially free of Oleuropein and containing about 25% Hydroxytyrosol.

### BRIEF DESCRIPTION OF THE SINGLE DRAWING FIGURE

The appended drawing FIGURE illustrates a preferred method of obtaining Hydroxytyrosol from olive leaves in a dry powder product containing about 25% Hydroxytyrosol.

### DETAILED DESCRIPTION OF THE PREFERRED EMBODIMENT

Turning now to the single drawing FIGURE, it is seen that at step 1, cultivated olive leaves are collected as a starting material for the process to be further described below. At step 2, pure, room temperature water is utilized along with agitation to wash the olive leaf starting materials. This water-wash and agitation step is sufficient to free the olive leaves of impurities and surface contaminants. This washing step may be repeated more than once if desired. However, experience has shown that a single water wash with agitation and subsequent draining of the wash water is sufficient to provide a clean olive leave material for further processing.

Next, at step 3, a 95% ethanol solution at a temperature in the range from about 75° C. to about 78° C. is utilized to soak the olive leaf starting material for at least 3 hours, and the liquid carrying extracted materials including Hydroxytyrosol and Oleuropein is drained and collected. This soaking in ethanol solution is repeated, and the two batches of drained liquid carrying extracted materials are combined (step 4). The liquid carrying extracted materials then receives further processing as described below, and the olive leaf mass remaining after the draining away of the ethanol solution is discarded. Alternatively this soaked olive leaf mass may be used for composting or other purposes.

Step 5 indicates a vacuum concentration step (i.e., “decompress” concentration), utilizing a temperature preferably below 60° C. (140° F.) to remove excess water, and reduce the volume of the extracted materials by about half (identified on the flow chart of the drawing FIGURE as “Concentrated Liquid”). Preferably, the temperature for this step is controlled in the range from about 59° C. to about 60° C. In other words, this step removes excess moisture by utilization of a relatively low temperature combined with application of a partial vacuum to draw off excess moisture by evaporation. At step 6 is defied a centrifugation step, to remove particulates and other undesired contaminants, resulting in a so called “clear liquid.” The centrifuging may be carried out as a batch process, but preferably a continuous flow type of centrifuge is utilized at this step.

In order to remove the ethanol from the clear liquid, step 7 indicates utilization of a resin column refining step, in which the clear liquid is run through a resin column in order to remove the ethanol, resulting in a “refined liquid” being produced. After this step, the resin column is regenerated to remove and collect the ethanol for later use.

Step 8 indicates the addition of a Bio-enzyme: Beta glycosidase enzymes, followed by a fermentation interval (Step 9) to product a “Fermented Concentrate Liquid.” At step 10 is indicated a continuation of the fermentation process, but now using the Fermented Concentrated Liquid, which still includes Oleuropein, adding about 0.08% to about 0.2% (V/V—i.e., volume-to-volume ratio) Biological invertase. Stirring is maintained, during an interval of about 5 hours, at a temperature of about 60° C.±2° C. The Applicant's preferred Fermentation and Biochemical incubator for use in these steps is generally know as a “SHP reaction pot,” which is simply a type of biochemical incubator.

Step 10 results in the production of a “Biotransformed Liquid” in which the Hydroxytyrosol originally extracted from the olive leaves is still present, but also in which Oleuropein extracted from the olive leaves has now been essentially completely converted to additional Hydroxytyrosol. Step 11 indicates that the Biotransformed Liquid is first subjected to a resin column purification, producing a “Purified Liquid” which is then filtered (Step 12) to provide a “Filtered Liquid.”

At step 13, the Filtered Liquid is spray dried, producing a dry crystalline, granular, intermediate product including about 25% (more-or-less, allowing for batch-to-batch variability) Hydroxytyrosol. As is indicated on the process flow chart of the drawing FIGURE, all that remains is the grinding (to reduce any clumps or cakes to a uniform granular product), testing (for % of Hydroxytyrosol, among other tests) blending of batches (i.e., to even the % of Hydroxytyrosol) and packaging (step 14).

Having considered the novel way the Applicant obtains the present product, it is well to consider how this product may be advantageously utilized, by way of example only, and not as a limitation on the present invention. Accordingly, it is to be understood that the present inventive product may be utilized as a daily dietary supplement, in a therapeutic dose range from about 50 mg to about 1000 mg of the dry powder product. This product may be mixed into food or taken with beverages, for example. Alternatively, the preferred daily dose may be taken in a soft gel capsule, with the product mixed into olive oil, or other oils, for example. Or, the dry powder product may be conveniently pressed into tablets, possibly including a binder to yield tablets of sufficient mechanical stability that they can be transported and handled well. The most preferred daily dietary supplemental dose is 100 mg. Still alternatively, the present inventive product may be utilized for cosmetic and skin care formulations. A user may benefit from use of a skin lotion, body lotion, moisturizing night cream, lip stick, skin treatment masque, sun screen or hair care product including the present product in the preferred range of from about 0.1% to perhaps 10.0%. The most preferred concentration of the present product in such cosmetic and skin care products is 1% to 2%.

The above detailed description of the invention is set forth solely to assist in understanding the invention. It is to be understood that variations of the invention, including all equivalents now known or later developed, are to be considered as falling within the scope of the invention, which is limited only by the following claims.

1. A method for obtaining Hydroxytyrosol from olive leaves in order to provide a dry powder product containing about 25% Hydroxytyrosol, said method comprising steps of:
collecting cultivated olive leaves;
utilizing room temperature water and agitation to wash the olive leaves so that they are substantially free of impurities and surface contaminants, and draining the wash water;
conducting a first soaking of the clean olive leaves in a 95% ethanol solution at a temperature in the range from about 75° C. to about 78° C. for about 3 hours, obtaining a first liquid carrying extracted materials including Hydroxytyrosol and Oleuropein, and draining and collecting this first liquid;
conducting a second soaking of the clean olive leaves previously soaked according to the preceding step, also in a 95% ethanol solution at a temperature in the range from about 75° C. to about 78° C. for about 3 hours, obtaining a second liquid carrying extracted materials including Hydroxytyrosol and Oleuropein, and draining and collecting this second liquid combined along with the first liquid obtained according to the preceding step;
performing a partial-vacuum concentration on the combined first and second liquid obtained in the soaking steps previously described, utilizing a temperature below 60° C. to remove excess water, and reduce the volume of the extracted materials by about half;
centrifuging the concentrated liquid obtained from the prior step, to remove particulates and other undesired contaminants, resulting in a “clear liquid;”
utilizing of a resin column refining to remove the ethanol from the clear liquid, resulting in a “refined liquid;”
adding a bio-enzyme: Beta glycosidase to the refined liquid, and allowing a fermentation interval, to product a “Fermented Concentrate Liquid;” adding about 0.08% to about 0.2% (V/V—i.e., volume-to-volume ratio) biological invertase to the fermented concentrated liquid, and stirring during an interval of about 5 hours, at a temperature of about 60° C.±2° C.; resulting in the production of a “Biotransformed Liquid,” in which the Hydroxytyrosol is present, but in which Oleuropein extracted from the olive leaves has been completely converted to additional Hydroxytyrosol;
subjecting the biotransformed liquid to a resin column purification, producing a “Purified Liquid;
filtering the purified liquid to provide a “Filtered Liquid;” and
spray drying the filtered liquid to provide a dry crystalline, granular, intermediate product including about 25% Hydroxytyrosol.

2. A method for extracting Hydroxytyrosol from olive leaves in order to provide a dry powder product containing about 25% Hydroxytyrosol, said method comprising steps of:
soaking olive leaves in a solution of ethanol to obtain liquid carrying extracted materials including Hydroxytyrosol and Oleuropein;
concentrating the liquid carrying extracted materials;
adding a bio-enzyme: Beta glycosidase to the liquid carrying extracted material; and
fermenting the liquid with added bio-enzyme;
adding biological invertase to the fermented liquid, and stirring during an interval to effect biotransformation of Oleuropein in the liquid carrying extracted material entirely into Hydroxytyrosol, producing a liquid free of Oleuropein;
purifying the extracted liquid free of Oleuropein to provide a purified liquid,
spray drying the purified liquid to produce a powder form product including about 25% Hydroxytyrosol.

3. The method of claim 2, wherein cultivated olive leaves are collected prior to said soaking step.

4. The method of claim 2, further including the step of utilizing water and agitation to wash the collected olive leaves so that they are substantially free of impurities and surface contaminants, and draining the wash water prior to the soaking step.

5. The method of claim 2, further including the steps of conducting a first soaking of the washed olive leaves in a 95% ethanol solution at a temperature in the range from about 75° C. to about 78° C. for about 3 hours, obtaining a first liquid carrying extracted materials including Hydroxytyrosol and Oleuropein, and draining and collecting this first liquid; and
conducting a second soaking of the clean olive leaves previously soaked according to the preceding step, also in a 95% ethanol solution at a temperature in the range from about 75° C. to about 78° C. for about 3 hours, obtaining a second liquid carrying extracted materials including Hydroxytyrosol and Oleuropein, and
draining and collecting the second liquid combined along with the first liquid obtained according to the preceding step.

6. The method of claim 2, further including the step of performing a partial-vacuum concentration on the combined first and second liquid obtained in the soaking steps previously described at a temperature below about 60° C. to remove excess water, and reduce the volume of the liquid carrying extracted materials by about half.

7. The method of claim 2, further including the steps of centrifuging the partial-vacuum-concentrated liquid to remove particulates and other undesired contaminants, resulting in a “clear liquid;” and
utilizing of a resin column refining to remove the ethanol from the clear liquid, resulting in a “refined liquid.”

8. The method of claim 7 further including after the step of adding bio-enzyme: Beta glycosidase to the refined liquid, the step of allowing a fermentation interval, to product a “Fermented Concentrate Liquid;” and
adding about 0.08% to about 0.2% (V/V—i.e., volume-to-volume ratio) biological invertase to the fermented concentrated liquid, and stirring during an interval of about 5 hours, at a temperature of about 60° C.±2° C.; resulting in the production of a “Biotransformed Liquid;” providing the biotransformed liquid in which the Hydroxytyrosol is present, but in which Oleuropein extracted from the olive leaves has been essentially completely converted to additional Hydroxytyrosol.

9. The method of claim 8 further including the steps of subjecting the biotransformed liquid to a resin column purification, producing a “Purified Liquid; and filtering the purified liquid to provide a “Filtered Liquid.”

10. The method of claim 9 further including the step of spray drying the filtered liquid to provide a dry crystalline, granular, intermediate product including about 25% Hydroxytyrosol.

11. The method of claim 9 further including the step of administering the product including about 25% Hydroxytyrosol as a daily dietary supplement for human consumption, administering daily a therapeutic dose in the range from about 50 mg to about 1000 mg of the dry powder product.

12. The method of claim 9 further including the step of administering the product including about 25% Hydroxytyrosol as a daily dietary supplement for human consumption, administering daily a therapeutic dose of substantially 100 mg of the dry powder product.

13. (canceled)

14. The method of claim 9 further including the step of pressing the product including about 25% Hydroxytyrosol into tablets.

15. (canceled)

16. (canceled)

17. (canceled)

18. A method for extracting Hydroxytyrosol from olive leaves in order to provide a dry powder product containing about 25% Hydroxytyrosol, said method comprising in order the steps of:
soaking olive leaves in a solution of ethanol to obtain a liquid carrying extracted materials including Hydroxytyrosol and Oleuropein;
concentrating the liquid carrying extracted materials to reduce its volume;
adding bio-enzymes to the concentrated liquid carrying extracted materials, and
utilizing the bio-enzymes to effect fermentation of the concentrated liquid carrying extracted materials;
adding biological invertase, and utilizing the biological invertase in a bio-reactor to effect transformation of the Oleuropein in the liquid entirely into Hydroxytyrosol;
spray drying the liquid resulting from the prior step to produce a dry powder product completely free of Oleuropein and containing about 25% Hydroxytyrosol.
